(19) United States
(12) Patent Application Publication
(10) Pub. No.: US 2014/0031632 A1
(43) Pub. Date: Jan. 30, 2014

(54) RETRACTOR

(71) Applicant: J. Morita Manufacturing Corporation, Kyoto (JP)

(72) Inventor: Masakazu Nakao, Kyoto (JP)

(73) Assignee: J. MORITA MANUFACTURING CORPORATION, Kyoto (JP)

(21) Appl. No.: 14/042,296

(22) Filed: Sep. 30, 2013

Related U.S. Application Data
(63) Continuation of application No. PCT/JP2011/001828, filed on Mar. 28, 2011.

(51) Int. Cl. A61B 17/02

(52) U.S. Cl. CPC A61B 17/0206
USPC 600/206

(57) ABSTRACT

A retractor includes a central fixed portion, an extended arm portion extending on both sides of the central fixed portion, and a hook support section which is attached to the extended arm portion so as to be slidable in a longitudinal direction and supports a retractor hook is formed of a plastically deformable composite member for, when receiving a deformation load of causing a bending deformation, permitting the extended arm portion to be plastically deformed in an in-plane direction Fh in a virtual plane F, which is formed of the central fixed portion and the extended arm portion, while restricting the extended arm portion regarding the deformation in an out-of-plane direction Fv.

### TECHNICAL FIELD

The present invention relates to a retractor used for, for example, heart surgery and the like, and in more detail, to a retractor having an accessory such as a retractor hook or the like mounted thereon.

### BACKGROUND ART

For example, in surgery of incising a body site such as heart surgery or the like, various retractors are used for enlarging the visual field of surgical procedure in the incised area. For example, a retractor as described in Patent Document 1 has been proposed.

For example, the retractor described in Patent Document 1 is used with a rib spreader, and has the following structure. On top of the rib spreader adjustable to be wider or narrower to suit to the size of the area of surgical procedure, a flexible U-shaped spring-like rail having a circular cross-section is provided, and retractor hook support means is movably supported by the spring-like rail. It is described that owing to such a structure, a desired retracted state is realized.

However, in the case of this retractor, the size of the opening between both ends of the U-shaped spring-like rail is adjusted by fastening or loosening a handle. Due to the property of the U-shaped spring-like rail, a loading force constantly acts in a direction to increase the width of the rib spreader. Therefore, in order to decrease the width of the rib spreader, the handle needs to be strongly fastened and fixed. If the fastened state of the handle is loosened, the U-shaped spring-like rail is inadvertently widened, which involves a danger of damaging the incised area.

### CITATION LIST

### Patent Literature

### [PTL 1]

Japanese PCT National-Phase Laid-Open Patent Publication No. 2000-507146

### SUMMARY OF INVENTION

### Technical Problem

The present invention has an object of providing a retractor for realizing a stable retracted state with a small number of operations.

### Solution to Problem

The present invention is directed to a retractor, comprising a holder main body; an arm portion extending on both sides of the holder main body; and a hook support section, attached to the arm portion so as to be slidable in a longitudinal direction, for supporting a retractor hook; wherein in at least a part of at least one of the holder main body and the arm portion, in-plane plastic deformation means is provided for permitting plastic deformation in an in-plane direction in a virtual plane formed of the holder main body and the arm portion while restricting deformation in an out-of-plane direction, when receiving a deformation load for causing a bending deformation.

The holder main body may be, for example, a portion which has the same cross-sectional shape as that of the arm portion and is integrally formed with the atm portion, or a fixing table attachable to the rib spreader or the like.

The bending deformation may mean curving deformation or folding deformation of the aim portion with respect to the holder main body caused by the hands or fingers, or curving deformation or folding deformation of the arm portion itself.

The in-plane plastic deformation means may be found of a member which forms at least a part of at least one of the holder main body and the arm portion, or a mechanism included in such a member. The “at least a part of at least one of the holder main body and the arm portion” means the entirety of, a part of, a plurality of parts of, both of the holder main body and the arm portion or either one of the holder main body and the arm portion.

The virtual plane formed of the holder main body and the arm portion is a phantom plane which includes the holder main body and the arm portion in the case where the holder main body and the arm portion are formed to have a shape of inverted letter C with two right-angled corners, letter U, letter C, L-shaped or any other desired shape. Accordingly, when the virtual plane is, for example, horizontal, the in-plane plastic deformation means for permitting the plastic deformation in the in-plane direction in the virtual plane while restricting the deformation in the out-of-plane direction is means for permitting the plastic deformation in the horizontal direction of the arm portion with respect to the holder main body. “Restricting the deformation in the out-of-plane direction” represents a concept of permitting a smaller deformation in the out-of-plane direction than the deformation in the in-plane direction.

Owing to this structure, a stable retracted state can be realized by a small number of operations.

In more detail, in at least a part of at least one of the holder main body and the arm portion, the in-plane plastic deformation means is provided. Therefore, the arm portion can be plastically deformed easily to a desired position merely by pushing or pulling the arm portion extending on both sides of the holder main body with fingers or hands to provide the arm portion with a deformation load for causing a bending deformation. Hence, the widening of the arm portion can be adjusted in accordance with the size of the area of surgical procedure, and the incised area is fixed in a widened state realized by the retractor hook supported by the hook support section attached to the arm portion so as to be slidable in the longitudinal direction. Thus, the visual field of surgical procedure can be enlarged.

By the in-plane plastic deformation means, the incised area is fixed in a widened state by the retractor hook supported by the hook support section attached to the arm portion plastically deformed with respect to the holder main body. Therefore, a stable retracted state can be kept.

As an embodiment of the present invention, the arm portion may be formed to have a circular cross-section; and the hook support section may include an attachment insertion hole for permitting the arm portion to be attached thereto so as to be rotatable in a circumferential direction around a center of the circular cross-section, and position fixing means for fixing the arm portion at a desired rotation position and a desired slide position.

Owing to this structure, the retractor hook can be fixed at a desired slide position and a desired rotation angle to the arm portion adjusted to be widened as desired.

In more detail, the arm portion is formed to have a circular cross-section, and the hook support section includes the attachment insertion hole which permits the hook support section to be attached to the aim portion such that the hook support section is rotatable in a circumferential direction around the center of the circular cross-section. Therefore, the hook support section can be attached at a desired rotation angle to the arm portion having a circular cross-section, and can be fixed by the position fixing means. Accordingly, the retractor hook can be fixed at an appropriate angle and an appropriate slide position and applied to retracting surgery regardless of the size of the target of surgical procedure, namely, whether the patient is a child or an adult. In addition, the arm portion extends on both sides of the holder main body. Therefore, the hook support section having a desired retractor hook attached thereto can be easily detached/attached or added from or to the arm portion from the open ends on both sides of the holder main body.

As an embodiment of the present invention, the hook support section may include an insertion hole for permitting a base shaft included in the retractor hook to be inserted therethrough; and the position fixing means may fix the base shaft inserted through the insertion hole.

Owing to this structure, a desired retractor hook can be easily attached. In more detail, the hook support section includes the insertion hole which permits the base shaft included in the retractor hook to be inserted therethrough, and the base shaft inserted through the insertion hole can be fixed by the position fixing means. Therefore, a desired retractor hook can be supported by the hook support section and the hook support section can be fixed by the position fixing means to the arm portion, and the base shaft inserted through the insertion hole can also be fixed by the position fixing means. Accordingly, the slide position and the rotation angle of the hook support section with respect to the arm portion, and the position in the axial direction and the orientation of the retractor hook with respect to the hook support section, can be fixed by a small number of operations, i.e., the fixing operation performed by the position fixing means.

As an embodiment of the present invention, the in-plane plastic deformation means may be formed of a plastically deformable composite member including a plastically deformable portion which is located at an inner position in the in-plane direction of the circular cross-section of the arm portion and has a cross-section thicker in the out-of-plane direction than in the in-plane direction; and an outer portion which is located outer to the plastically deformable portion in the in-plane direction of the circular cross-section and has a higher bending performance than the plastically deformable portion; and the in-plane plastic deformation means may form at least a part of at least one of the holder main body and the arm portion.

The plastically deformable portion may be formed of a metal material which is plastically deformable repeatedly at room temperature such as nickel titanium or the like, so-called flexible steel, a shape memory alloy or a resin which is plastically deformable repeatedly.

The outer portion may be formed of a resin or a metal material which has a higher bending performance than the plastically deformable portion and can be integrated with the plastically deformable portion by attachment, welding or the like to have a circular cross-section, and further, a resin or a metal material having durability against autoclave such as, for example, silicone resin or the like.

Therefore, the plastically deformable composite member may be formed of any of various combinations, for example, a plastically deformable metal-resin composite member which forms a sandwich structure in the in-plane direction by a metal plastically deformable portion and a resin outer portion, a plastically deformable metal-metal member formed of a metal plastically deformable portion and a metal outer portion, a plastically deformable resin-resin member formed of a resin plastically deformable portion and a resin outer portion, or a plastically deformable resin-metal composite member formed of a resin plastically deformable portion and a metal outer portion.

The “inner” in the in-plane direction of the circular cross-section of the arm portion means the inner side in the direction in which the parts of the sandwich structure are stacked.

The “outer” in the in-plane direction mentioned regarding the plastically deformable portion of the circular cross-section means the outer side in the direction in which the parts of the sandwich structure are stacked.

Owing to this structure, the in-plane plastic deformation means for permitting the plastic deformation in the in-plane direction in the virtual plane formed of the holder main body and the arm portion while restricting the deformation in the out-of-plane direction when receiving a deformation load for causing a bending deformation can be provided even without a complicated mechanism.

In more detail, at least a part of at least one of the holder main body and the arm portion is formed of a plastically deformable composite member, which includes a plastically deformable portion that is located at an inner position of the in-plane direction of the circular cross section of the arm portion and has a larger thickness in the out-of-plane direction than in the in-plane direction, and an outer portion which is located outer to the plastically deformable portion in the in-plane direction of the circular cross section and has a higher bending performance than the plastically deformable portion. Owing to this, the plastically deformable portion having a larger thickness in the out-of-plane direction than in the in-plane direction can permit the plastic deformation in the in-plane direction in the virtual plane while restricting the deformation in the out-of-plane direction.

Accordingly, as compared with the case where the in-plane plastic deformation means includes a complicated mechanism, the in-plane plastic deformation means is lightweight and the function stability as the in-plane plastic deformation means can be improved. In addition, the in-plane plastic deformation means does not include any inner mechanism. Therefore, also as compared with the case where the in-plane plastic deformation means includes a complicated mechanism, the effect of sterilization processing by autoclave or the like can be improved.

### Advantageous Effects of Invention

Owing to the present invention, a retractor for realizing a stable retracted state with a small number of operations can be provided.

### BRIEF DESCRIPTION OF DRAWINGS

FIG. 1 is an isometric view of a retractor unit.

FIGS. 2A and 2B illustrate a support arm.

FIG. 3 illustrates a virtual plane.

FIG. 4 is a cross-sectional view of a fixing table.

FIG. 5 is a cross-sectional view of a hook support section.

FIG. 6 illustrates attachment and fixation of the hook support section.

FIG. 7 illustrates adjustment of the attaching position of the retractor hook to the hook support section.

FIG. 8 illustrates adjustment of the retractor hook by rotation.

FIG. 9 is a plan view of the retractor unit.

FIG. 10 is a plan view of the retractor unit.

### DESCRIPTION OF EMBODIMENTS

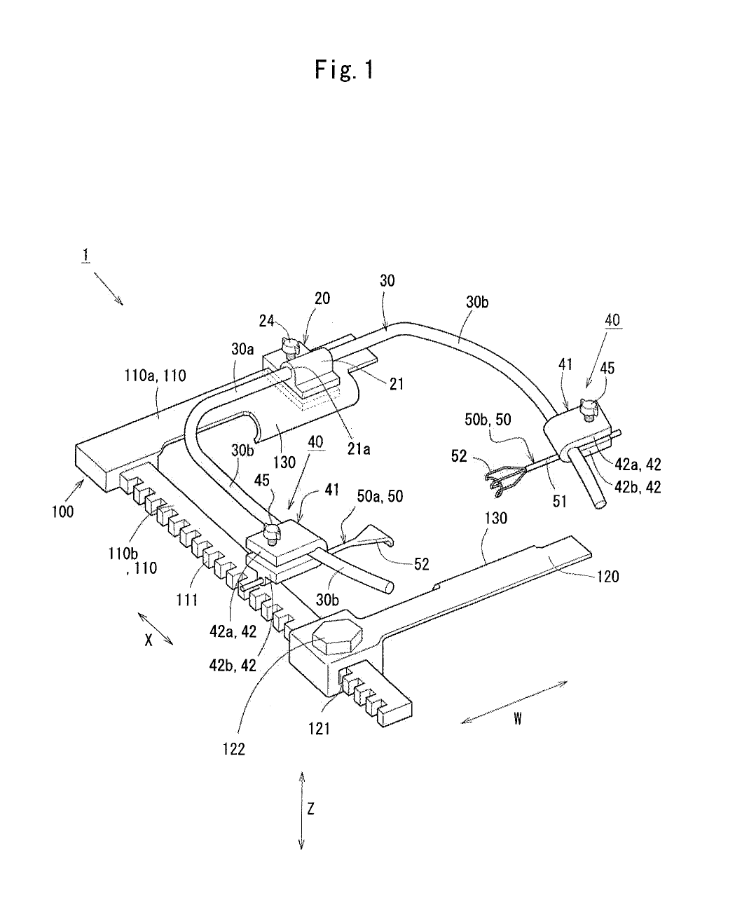

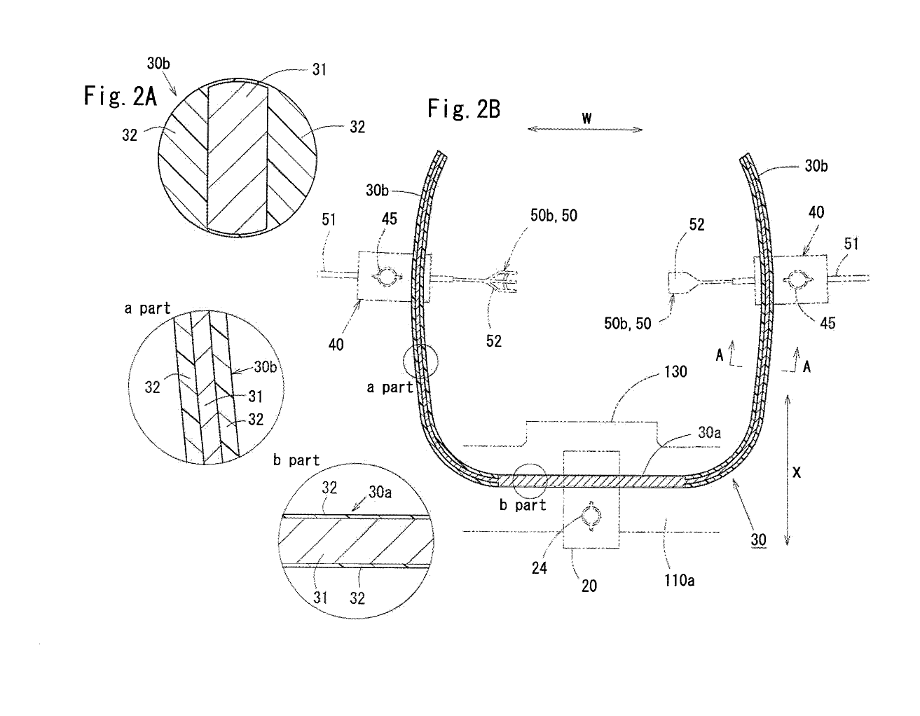

An embodiment of the present invention will be described with reference to the drawings. FIG. 1 is an isometric view of a retractor unit 1, and FIGS. 2A and 2B illustrate a support arm 30. In more detail, FIG. 2A is a cross-sectional view of an extended aim portion 30b shown in FIG. 2B taken along line A-A in FIG. 2B, and FIG. 2B is a cross-sectional view of the support arm 30 taken in a planar direction. The circled figures in FIG. 2B labeled “a” and “b” are respectively enlarged views of the “a” part and the “b” part in FIG. 2B.

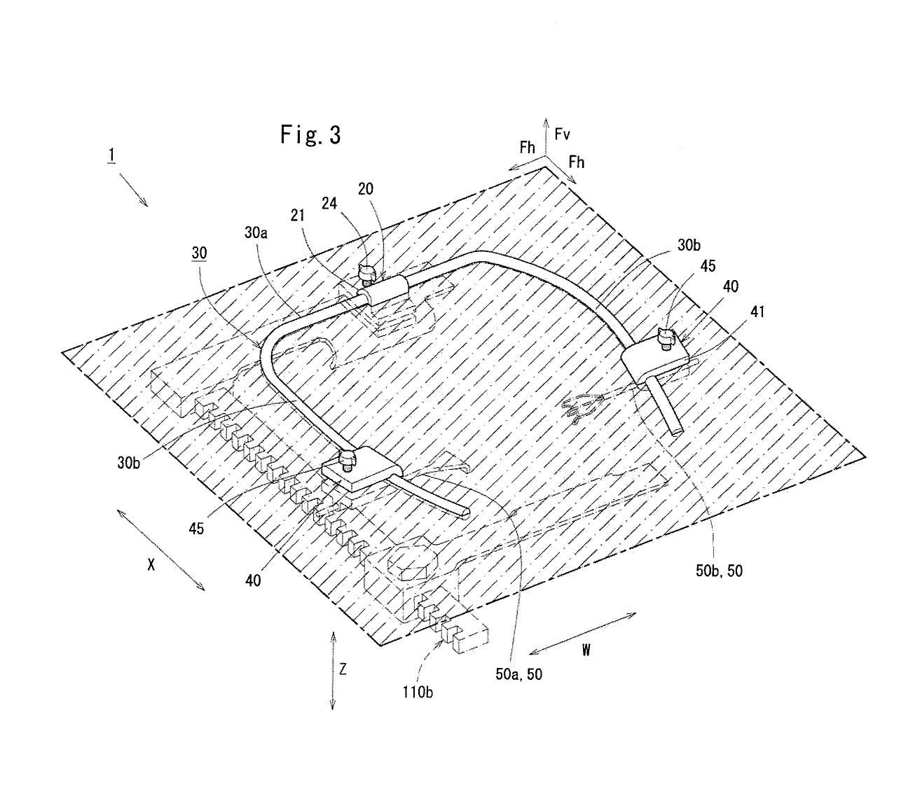

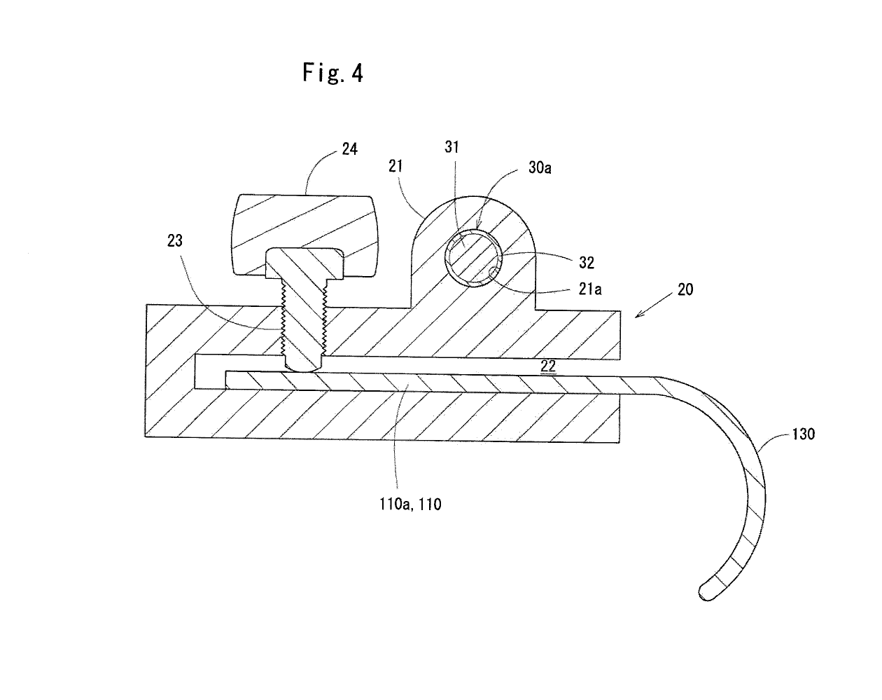

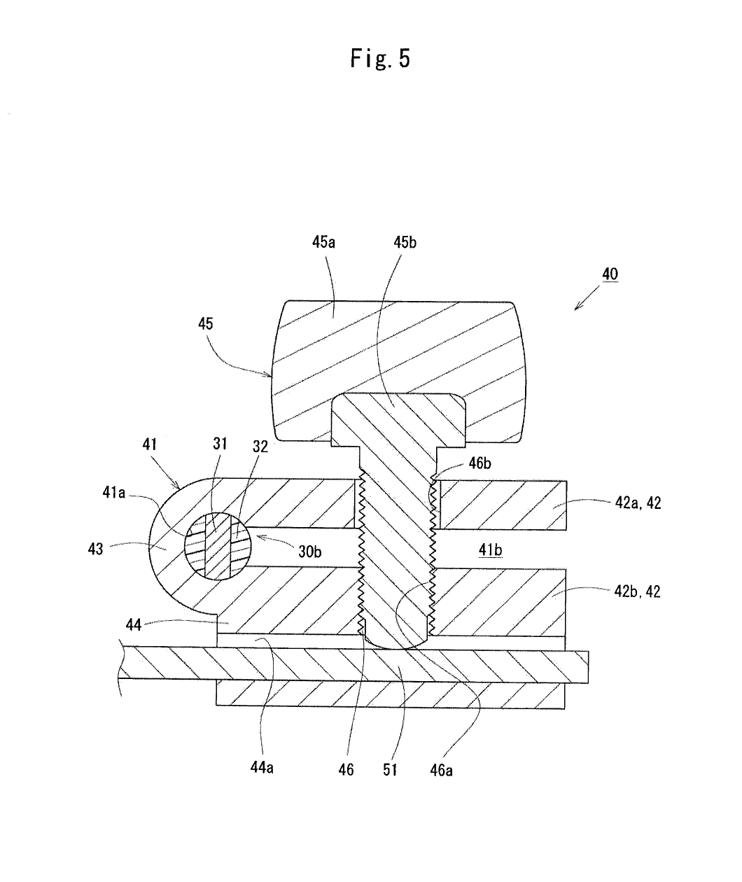

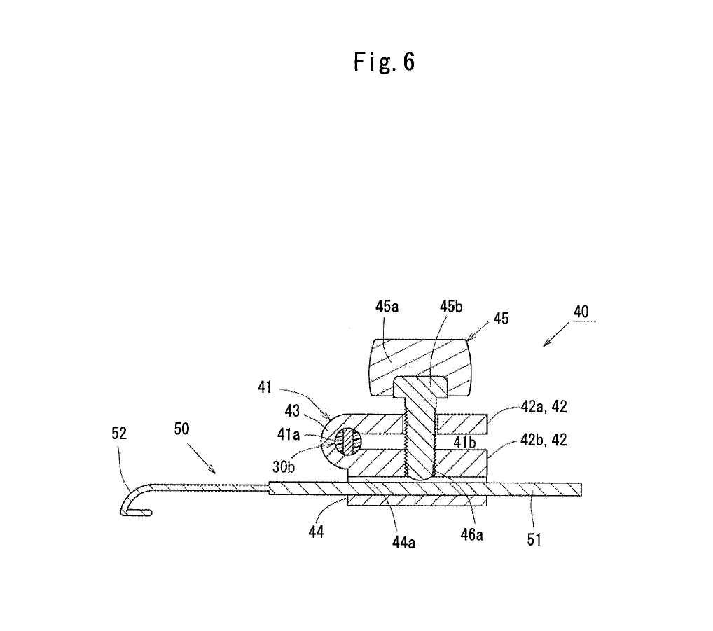

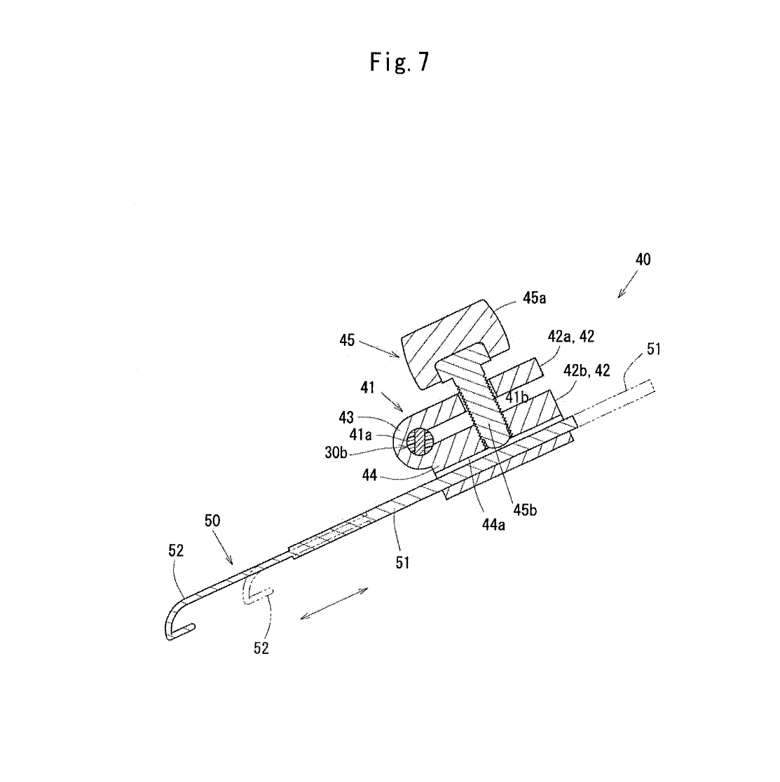

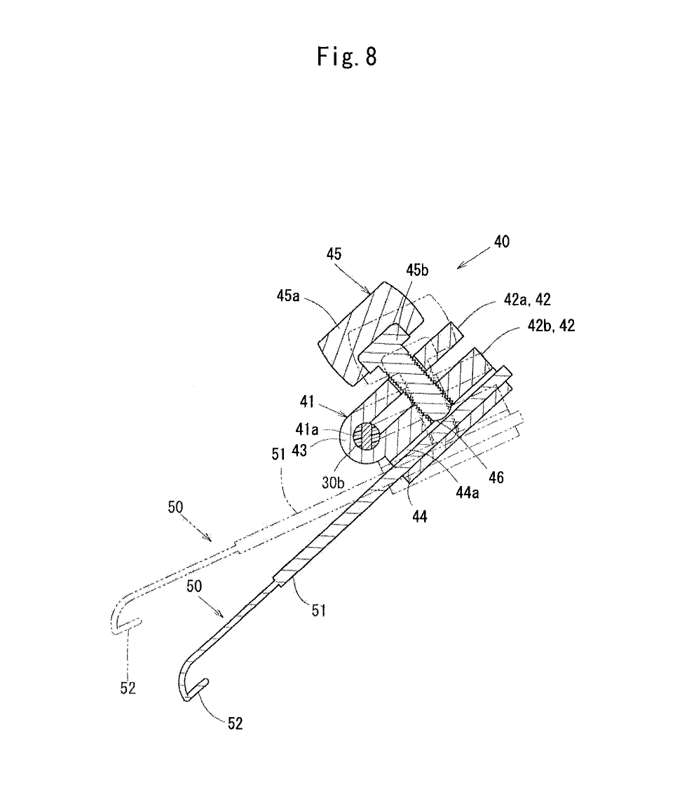

FIG. 3 is an isometric view illustrating a virtual plane F, FIG. 4 is a cross-sectional view of a fixing table 20, and FIG. 5 is a cross-sectional view of a hook support section 40. FIG. 6 is a side view illustrating attachment and fixation of the hook support section 40, FIG. 7 is a side view illustrating adjustment of the attaching position of the retractor hook 50 to the hook support section 40, and FIG. 8 is a side view illustrating adjustment of the retractor hook 50 by rotation.

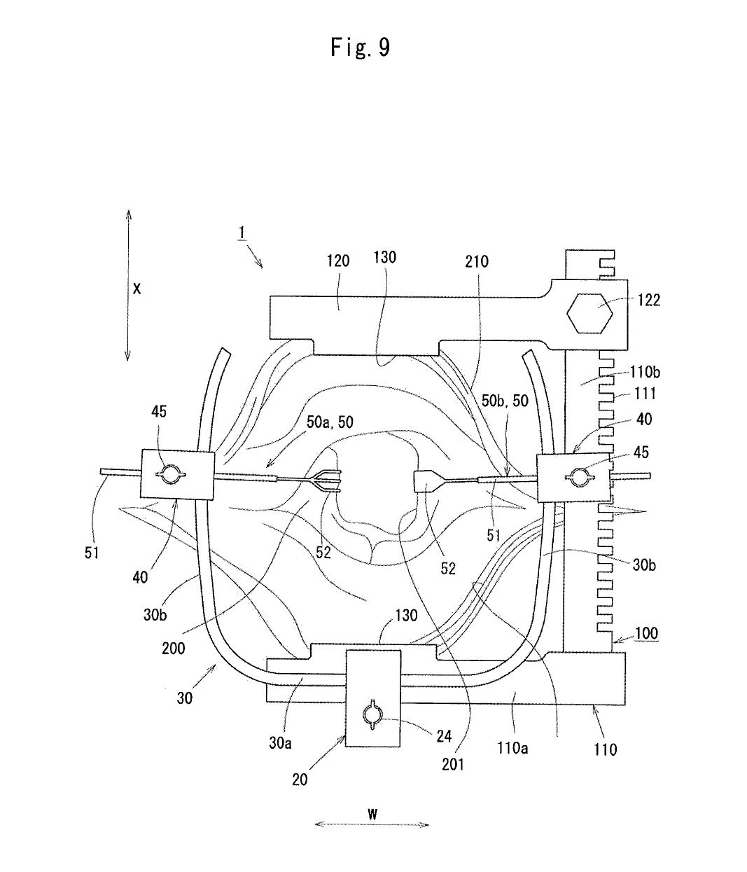

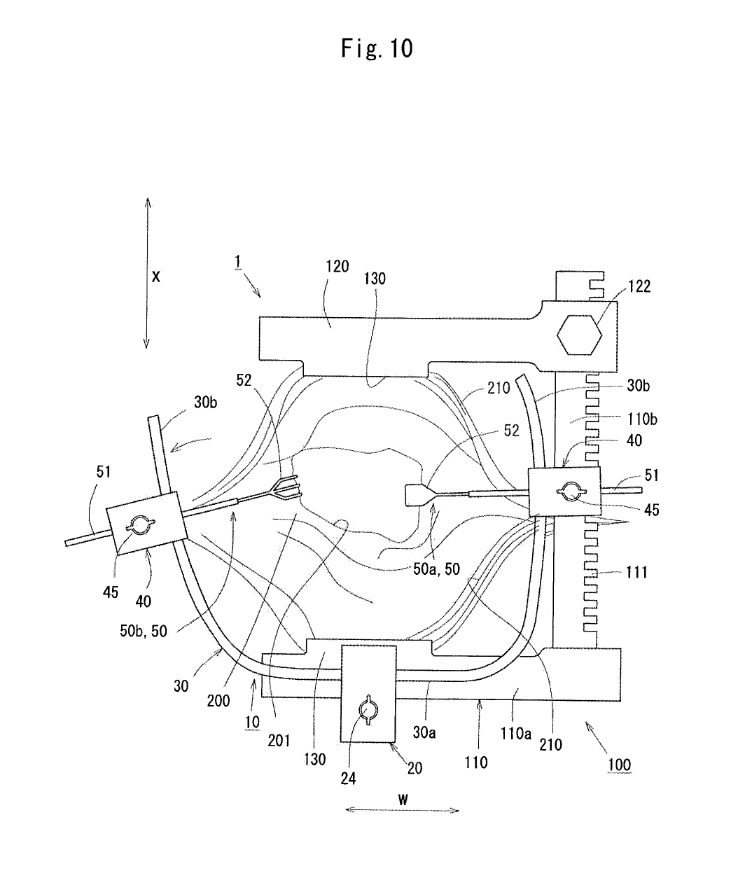

FIGS. 9 and 10 are plan views of the retractor unit 1. In more detail, FIG. 9 is a plan view of a state where a surgery area 210 is opened by a rib spreader 100, and FIG. 10 is a plan view of a state where an incised area 201 of the sternum 200, which is a target of surgical procedure, by the retractor hook 50 of a retractor 10.

The retractor unit 1 includes the rib spreader 100 which has a shape of letter C with two right-angled corners as seen in a plan view, and the retractor 10 fixed on top of the rib spreader 100.

The rib spreader 100 includes an L-shaped base frame 110 and a slide frame 120 slidable in a depth direction X with respect to the base frame 110. The base frame 110 includes a core frame 110a extending in a width direction W and a rail frame 110b extending perpendicular to the core frame 110a, namely, in the depth direction X. Thus, the base frame 110 is L-shaped as seen in a plan view. An outer side surface of the rail frame 110b in the width direction W has a concaved and convexed part 111. Convexed portions of the concaved and convexed part 111 externally protrude in the width direction W.

The slide frame 120 located parallel to the core frame 110a of the base frame 110 has a frame insertion hole 121, on one end thereof, which allows the rail frame 110b to be inserted therethrough, and also includes a slide position adjusting knob 122 projecting above the frame insertion hole 121.

In more detail, in the frame insertion hole 121, the slide position adjusting knob 122 projecting above the frame insertion hole 121 is engaged with the concaved and convexed part 111 of the rail frame 110b inserted through the frame insertion hole 121. By rotating the slide position adjusting knob 122, the position of the slide frame 120 in the depth direction X with respect to the rail frame 110b can be adjusted.

The core frame 110a and the slide frame 120 are each like a plate having a small thickness in a height direction Z, and each include a stopping blade 130, protruding internally in the depth direction X, in the vicinity of the center in the width direction W.

The retractor 10 includes a fixing table 20 for attaching and fixing the retractor 10 to the core frame 110b of the rib spreader 100, the support arm 30, and the hook support section 40. The retractor hook 50 is attachable to the hook support section 40.

As shown in FIGS. 1 and 4, the fixing table 20 has a parallelepiped shape which is longer in the depth direction X and is low in the height direction Z. The fixing table 20 includes an arm fixing portion 21 protruding from a top surface thereof. The arm fixing portion 21 has an arm fixing hole 21a which allows a central fixed portion 30a of the support arm 30 described later to be inserted therethrough and fixed. The fixing table 20 also has a fitting groove 22 which allows the core frame 110a to be fit thereinto in the depth direction X. The fitting groove 22 is recessed from a front side toward a rear side of the fixing table 20.

Rearward in the depth direction X to the arm fixing portion 21 of the fixing table 20, an attaching and fixing bolt 24 is provided to be screwed from the top surface of the fixing table 20 into a vertical screw hole 23 which is communicated to the inside of the fitting groove 22. The central fixed portion 30a inserted through the arm fixing hole 21a of the aim fixing portion 21 is fixed to the arm fixing portion 21. As a result, the fixing table 20 and the support arm 30 are integral.

As shown in FIGS. 2A and 2B, the support arm 30 includes the central fixed portion 30a which forms a holder main body, and the extended arm portion 30b extending from both ends of the central fixed portion 30a. Thus, the support arm 30 is generally U-shaped as seen in a plan view. The support arm 30 has a circular cross-section throughout the entire length thereof.

As shown in FIG. 2A and an “a” part enlarged view in FIG. 2B, the extended arm portion 30b includes a core metal part 31 of lengthy cross-section located at an inner position of the circular cross-section in the width direction W, and a circle-forming resin part 32 located on both sides of the core metal part 31 of lengthy cross-section in the width direction W, namely, an in-plane direction Fh. Namely, the extended aim portion 30b is formed of a plastically deformable composite member corresponding to in-plane plastic deformation means. The circled figures in FIG. 2B labeled “a” and “b” are respectively enlarged views of the “a” part and the “b” part.

In more detail, the core metal part 31 of lengthy cross-section corresponding to a plastically deformable part is located at an inner position of the circular cross-section in the width direction W and has a generally rectangular cross-section longer in the height direction Z, namely, an out-of-plane direction Fv.

As shown in FIGS. 2 and 3, the core metal part 31 of lengthy cross-section having a generally rectangular cross-section longer in the height direction Z, namely, the out-of-plane direction Fv of a virtual plane F passes through the central fixing section 30a and the extended arm portion 30b and is formed of a flexible steel which is plastically deformable repeatedly when receiving a bending deformation load in the in-plane direction Fh in the virtual plane F, which is formed of the width direction W and the depth direction X. As shown in FIG. 3, the in-plane direction Fh may be any of all directions on the virtual plane F, and the out-of-plane direction Fv is a direction crossing the virtual plane F.

The circle-forming resin part 32 corresponding to an outer portion is located on both outer sides of the core metal part 31 of lengthy cross-section in the width direction W, namely, in a direction in which the parts forming the circular cross-section are stacked to form a sandwich structure. The circle-forming resin part 32 is attached to side surfaces of the core metal part 31 of lengthy cross-section to be integral therewith to form the circular cross-section. The circle-forming resin part 32 is formed of a resin having durability against autoclave, for example, a silicone resin.

As described above, the extended arm portions 30b has the sandwich structure in the in-plane direction Fh as a result of a combination of the hard, plastically deformable core metal part 31 of lengthy cross-section and the circle-forming resin part 32 which is softer than the core metal part 31 of lengthy cross-section.

By contrast, the central fixed portion 30a is formed of the same metal as the core metal part 31 of lengthy cross-section in almost the entirety of the cross-section thereof. An outer circumferential surface of the metal is covered with the resin continued from the circle-forming resin part 32. Since the central fixed portion 30a is formed of the same metal as the core metal part 31 of lengthy cross-section in almost the entirety of the cross-section thereof, the central fixed portion 30a is restricted regarding the plastic deformation in the in-plane direction Fh in addition to the out-of-plane direction Fv in the virtual plane F, unlike the extended arm portions 30b formed by attaching the circle-forming resin part 32 to the side surfaces of the core metal part 31 of lengthy cross-section into an integral body.

The hook support section 40 is mounted on the extended arm portion 30b in the state where the sliding angle and the rotation angle of the hook support section 40 are adjustable. As shown in FIG. 1, the hook support section 40 includes a U-shaped pedestal 41 which has a generally U-shaped cross-section and is oriented such that an open end is directed externally with respect to the extended aim portion 30b of the support arm 30 formed to be generally U-shaped as seen in a plan view; a hook attaching portion 44 located on a bottom surface of the U-shaped pedestal 41 and having a shaft insertion hole 44a which runs through the hook attaching portion 44 in the width direction W, allows a base shaft 51 of the retractor hook 50 to be inserted therethrough, and corresponds to an insertion hole section; and a fixing and adjusting pinch bolt 45 running from a top surface of the U-shaped pedestal 41 to the shaft insertion hole 44a.

In more detail, as shown in FIG. 5, the U-shaped pedestal 41 includes two end portions 42 of the U-shaped pedestal separated from each other by a gap 41b and extending in a horizontal direction as seen in a front view (the depth direction X in FIG. 1 from the side of the opening of the support arm), and a U-shaped pedestal arc portion 43 for connecting the two end portions 42 of the U-shaped pedestal. The U-shaped pedestal arc portion 43 is semi-circular as seen in the front view. Thus, the U-shaped pedestal 41 is U-shaped as seen in the front view.

The U-shaped pedestal 41 has an arm insertion hole 41a inside the U-shaped pedestal arc portion 43. The arm insertion hole 41a allows the extended arm portion 30b to be inserted therethrough and fauns an insertion hole. The gap 41b between the two end portions 42 of the U-shaped pedestal (42a, 42b) has a height lower than the height of the arm insertion hole 41a.

As shown in FIG. 5, the hook attaching portion 44 is formed to have a rectangular shape longer in the width direction W as seen in the front view and is integral with the lower end portion 42b of the U-shaped pedestal, among the end portions 42 included in the U-shaped pedestal 41. The hook attaching portion 44 is formed such that the shaft insertion hole 44a allowing the base shaft 51 of the retractor hook 50 to be inserted therethrough is parallel to the end portions 42 of the U-shaped pedestal. The shaft insertion hole 44a has a slightly larger diameter than that of the base shaft 51.

The hook support section 40 has a through-hole 46 extending in the height direction Z, which is communicated from the U-shaped pedestal 41 to the shaft insertion hole 44a, communicates the lower end portion 42b of the U-shaped pedestal 41 to the hook attaching portion 44, and has a thread 46a. The upper end portion 42a of the U-shaped pedestal, among the end portions 42 of the U-shaped pedestal, has a through-hole 46b which has a slightly larger diameter than an outer diameter of a bolt 45b of the fixing and adjusting pinch bolt 45 described later.

The fixing and adjusting pinch bolt 45 co-operable with the thread 46a of the through-hole 46 to form position fixing means includes the bolt 45b and a pinch cap 45a mounted on a head of the bolt 45b. The fixing and adjusting pinch bolt 45 can be screwed with the thread 46a of the through-hole 46. By screwing the fixing and adjusting pinch bolt 45 through the through-hole 46, the top of the bolt 45b presses the upper end portion 42a of the U-shaped pedestal downward, namely, in such a direction as to eliminate the gap 41b; while a bottom end of the fixing and adjusting pinch bolt 45 protrudes into the shaft insertion hole 44a and can press the base shaft 51 inserted through the shaft insertion hole 44a.

The retractor hook 50 fixed by the fixing and adjusting pinch bolt 45 after the base shaft 51 is inserted through the shaft insertion hole 44a includes a hook portion 52 of any of various types in accordance with the target of surgical procedure or the retracting method. As shown in FIG. 1, the hook portion 52 may be a spatula type hook portion 50a which is generally spatula-shaped, or a fork type hook portion 50b which is generally fork-shaped. In any case, the hook portion 52 and the base shaft 51 are integrally formed.

The retractor unit 1 is formed by assembling the retractor 10, the retractor hook 50 and the rib spreader 100 structured as described above. With reference to FIG. 9 and FIG. 10, the assembly will be described below in detail.

First, the slide frame 120 is attached to the rail frame 110b to form the rib spreader 100 which is formed to have a shape of inverted letter C with two right-angled corners as seen in a plan view. The fitting groove 22 is fit into the core frame 110a of the rib spreader 100. The attaching and fixing bolt 24 is screwed into the fitting groove 22 to fix the fixing table 20 to the rib spreader 100.

The support aim 30 including the central fixed portion 30a inserted through, and thus fixed to, the arm fixing hole 21a of the arm fixing portion 21 of the fixing table 20 is integral with the fixing table 20. Therefore, by fixing the fixing table 20 to the rib spreader 100, the support arm 30 is also fixed to the rib spreader 100 via the fixing table 20. At this point, the opening direction of the rib spreader 100 having a shape of inverted letter C with two right-angled corners and the opening direction of the U-shaped support arm 30 are different from each other; namely, the opening between the core frame 110a and the slide frame 120 and the opening between the ends of the extended arm portion 30b are directed in different directions from each other.

The extended arm portion 30b of the support arm 30 fixed to the rib spreader 100 via the fixing table 20 in this manner is inserted through the arm insertion hole 41a to attach the hook support section 40 at a desired position of the extended aim portion 30b. Then, the base shaft 51 of the retractor hook 50 is inserted through the shaft insertion hole 44a of the hook attaching portion 44 to attach the retractor hook 50 to the hook attaching portion 44.

At this point, as shown in FIG. 6, by loosening the fixing and adjusting pinch bolt 45, the fixing and adjusting pinch bolt 45 is allowed to be screwed only to the middle of the through-hole 46. Therefore, the U-shaped pedestal 41 does not fasten the extended arm portion 30b. As shown in FIG. 8 with the two-dot chain line, the hook support section 40 is rotatable around the center of the cross-section of the extended aim portion 30b and is also slidable in a longitudinal direction of the extended arm portion 30b.

The fixing and adjusting pinch bolt 45 is screwed only to the middle of the through-hole 46. Therefore, the fixing and adjusting pinch bolt 45 is not fixed to the base shaft 51 inserted through the shaft insertion hole 44a, either. Thus, as shown in FIG. 7 with the two-dot chain line, the position in the axial direction and the orientation of the retractor hook 50 with respect to the hook attaching portion 44 can be adjusted.

In this state, the slide position in the longitudinal direction and the rotation angle of the hook support section 40 with respect to the extended arm portion 30b are made adjustable. Also, the position in the axial direction of the retractor hook 50 with respect to the hook attaching portion 44, namely, the extending amount and the orientation of the retractor hook 50 with respect to the hook attaching portion 44 are adjusted, and then the fixing and adjusting pinch bolt 45 is screwed to fix the slide position and the rotation angle of the hook support section 40 with respect to the extended arm portion 30b and also the position in the axial direction and the orientation of the retractor hook 50 with respect to the hook attaching portion 44. Thus, adjustment of the position of the retractor unit 1 is completed.

With reference to FIG. 9 and FIG. 10, a method for using the retractor unit 1 having such a structure will be described. In this embodiment, the method for using the retractor unit 1 in thoracotomy will be described, but the present invention is not limited to this and is applicable to a different surgical procedure site such as the belly or the like.

First, before using the retractor unit 1 in thoracotomy, the chest as the surgical procedure site is spread using a surgical tool such as a scalpel or the like, and the stopping blades 130 of the rib spreader 100 are inserted into the surgery area 210 of the spread surgical procedure site. In order to enlarge the visual field of surgical procedure in the surgery area 210, the slide position adjusting knob 122 of the slide frame 120 is rotated to widen the gap between the stopping blades 130 in the depth direction X and thus to widen the surgery area 210. In this state, the stopping blades 130 are fixed.

Next, the sternum 200 as the target of surgical procedure is incised, and the extended arm portion 30b is pushed or pulled in accordance with the incised area 201 which has been incised, so that a load for causing a bending deformation is acted on the extended arm portion 30b in the width direction W. As shown in FIG. 10, the extended arm portion 30b on which the bending load has been acted is plastically deformed in the in-plane direction Fh in the virtual plane F formed of the central fixed portion 30a and the extended arm portion 30b, which form the support arm 30. This plastic deformation of the extended arm portion 30b allows the incised area 201 of the organ to be held in a widened state using the retractor hook 50 or allows the organ to be held in a closed state.

When the angle or the position of the retractor hook 50 with respect to the extended aim portion 30b needs to be adjusted in accordance with the surgical procedure, the fixing and adjusting pinch bolt 45 is once screwed out, the slide position or the rotation angle of the hook support section 40 with respect to the extended atm portion 30b and the position in the axial direction or the orientation of the retractor hook 50 with respect to the hook attaching portion 44 are adjusted, and then the fixing and adjusting pinch bolt 45 is screwed again to fix the resultant state.

When another retractor hook 50 is needed, another hook support section 40 is attached from the open ends of the extended arm portion 30b and is fixed at a desired slide position and rotating angle. Thus, the retractor hook 50 is added.

Because the retractor 10 included in the retractor unit 1 has the above-described structure, a stable retracted state can be realized using the retractor hook 50 with a small number of operations.

Specifically, in the retractor 10 including the central fixed portion 30a, the extended atm portion 30b extending on both sides of the central fixed portion 30a, and the hook support section 40 which is attached to the central fixed portion 30a so as to be slidable in the longitudinal direction and supports the retractor hook 50, the extended arm portion 30b is formed of a plastically deformable composite member including the core metal part 31 of lengthy cross-section and the circle-forming resin part 32. Owing to this, when receiving a deformation load for causing a bending deformation, the extended arm portion 30b is permitted to be the plastically deformed in the in-plane direction Fh in the virtual plane F while being restricted regarding the deformation in the out-of-plane direction Fv.

In more detail, the extended arm portion 30b is formed of a plastically deformable composite member, which includes the core metal part 31 of lengthy cross-section that is located at an inner position of the in-plane direction Fh of the circular cross section and has a lengthy cross-section thicker in the in-plane direction Fh than in the out-of-plane direction Fv, and the circle-forming resin part 32 provided outer to the core metal part 31 of lengthy cross-section in the in-plane direction Fh of the circular cross section. Owing to this, the extended aim portion 30b can be plastically deformed easily to a desired position in the in-plane direction Fh in the virtual plane F while being restricted regarding the deformation in the out-of-plane direction Fv merely by pushing or pulling the extended arm portion 30b, extending on both sides of the central fixed portion 30a toward the open ends, with fingers of hands to provide the extended arm portion 30b with a deformation load for causing a bending deformation, even without any complicated mechanism.

In more detail, when the extended arm portion 30b is plastically deformed in the out-of-plane direction Fv in addition to the in-plane direction Fh, the surgical operator needs to adjust the plastic deformation of the extended aim portion 30b three-dimensionally in order to open the incised area 201. By contrast, the retractor 10 permits the plastic deformation of the extended portion 30b in the in-plane direction Fh while restricting the deformation thereof in the out-of-plane direction Fv, owing to the core metal part 31 of lengthy cross-section. Therefore, the surgical operator merely needs to adjust the plastic deformation of the extended aim portion 30b two-dimensionally. This improves the operability for the surgical operator and allows the extended arm portion 30b to be plastically deformed easily to a desired position.

Hence, the widening of the extended arm portion 30b can be adjusted in accordance with the size of the area of surgical procedure, and the incised area 201 is fixed in a widened state realized by the retractor hook 50 supported by the hook support section 40 attached to the extended arm portion 30b so as to be slidable in the longitudinal direction. Thus, the visual field of surgical procedure can be enlarged.

As described above, the extended aim portion 30b is formed of a plastically deformable composite member. Owing to this, the retracted state in which the incised area 201 is widened and fixed by the retractor hook 50 supported by the hook support section 40 attached to the extended arm portion 30b which is plastically deformed with respect to the central fixed portion 30a can be stabilized.

The extended arm portion 30b is formed of a plastically deformable composite member including the core metal part 31 of lengthy cross-section and the circle-forming resin part 32. Owing to this, the extended arm portion 30b is permitted to be plastically deformed in the in-plane direction Fh while being restricted regarding the deformation in the out-of-plane direction Fv. Therefore, the extended atm portion 30b is lightweight and the plastic deformation function thereof can be stabilized in the in-plane direction Fh in the virtual plane F, as compared with a case where the extended arm portion 30b has a complicated mechanism.

In addition, the extended arm portion 30b does not include any inner mechanism. Therefore, also as compared with a case where the extended arm portion 30b has a complicated mechanism, the effect of sterilization processing by autoclave or the like can be improved.

The extended arm portion 30b is formed to have a circular cross-section. The hook support section 40 includes the arm insertion hole 41a which permits the hook support section 40 to be attached to the extended min portion 30b such that the hook support section 40 is rotatable in a circumferential direction around the center of the circular cross-section, and also includes the fixing and adjusting pinch bolt 45 for fixing the hook support section 40 at a desired rotation position and a desired slide position. Owing to this, the retractor hook 50 can be fixed at a desired slide position and a desired rotation angle with respect to the extended aim portion 30b adjusted to be widened as desired.

In more detail, the extended arm portion 30b is formed to have a circular cross-section, and the hook support section 40 includes the arm insertion hole 41a which permits the hook support section 40 to be attached to the extended arm portion 30b such that the hook support section 40 is rotatable in a circumferential direction around the center of the circular cross-section. Therefore, the hook support section 40 can be attached at a desired rotation angle to the extended arm portion 30b having the circular cross-section and can be fixed by the fixing and adjusting pinch bolt 45.

Namely, the hook support section 40 is formed to have the angle thereof adjustable with respect to the extended arm portion 30b, which is restricted to be plastically deformed two-dimensionally in the in-plane direction Fh by the core metal part 31 of lengthy cross-section. Owing to this, the adjustment in the out-of-plane direction Fv in addition to the in-plane direction Fh, namely, the adjustment in three dimensions, is made possible. Accordingly, the retractor hook 50 can be fixed at an appropriate angle and an appropriate slide position and applied to retracting surgery regardless of the size of the target of surgical procedure, namely, whether the patient is a child or an adult.

The extended portion 30b extends on both sides of the central fixed portion 30a toward the open ends. Therefore, the hook support section 40 having a desired retractor hook 50 attached thereto can be easily attached/detached or added.

The extended arm portion 30b having a circular cross-section is inserted through the arm insertion hole 41a included in the hook support section 40 and fixed as being fastened by the fixing and adjusting pinch bolt 45. The extended arm portion 30b is formed of a plastically deformable composite member including the core metal part 31 of lengthy cross-section and the circle-forming resin part 32. Therefore, the fastened portion around the arm insertion hole 41a bites into the circle-forming resin part 32 which is softer than the core metal part 31 of lengthy cross-section. As a result, a firm fixing state can be realized.

The hook support section 40 has a shaft insertion hole 44a which permits the base shaft 51 included in the retractor hook 50 to be inserted therethrough, and the base shaft 51 inserted through the shaft insertion hole 44a is fixed by the fixing and adjusting pinch bolt 45. Therefore, a desired retractor hook 50 can be easily attached.

In more detail, the hook support section 40 includes the shaft insertion hole 44a which permits the base shaft 51 included in the retractor hook 50 to be inserted therethrough, and the base shaft 51 inserted through the shaft insertion hole 44a can be fixed by the fixing and adjusting pinch bolt 45. Therefore, a desired retractor hook 50 can be supported by the hook support section 40 and fixed by the fixing and adjusting pinch bolt 45 to the extended arm portion 30b, and the base shaft 51 inserted through the shaft insertion hole 44a can also be fixed by the fixing and adjusting pinch bolt 45.

Namely, by using the fixing and adjusting pinch bolt 45 in the hook support section 40, two types of fixation, i.e., the fixation of the slide position and the rotation angle of the hook support section 40 with respect to the extended arm portion 30b, and the fixation of the position in the axial direction and the orientation of the base shaft 51 with respect to the shaft insertion hole 44a, can be realized by one fixing operation. Accordingly, the slide position and the rotation angle of the hook support section 40 with respect to the extended arm portion 30b, and also the position in the axial direction and the orientation of the retractor hook 50 with respect to the hook support section 40, can be fixed with a small number of operations.

As described above, the retractor 10 is applicable to retracting surgery on a child or an adult regardless of the size of the area of surgical procedure by fixing the retractor hook 50 at an appropriate angle and an appropriate slide position. Moreover, the slide position and the rotation angle of the hook support section 40 with respect to the extended arm portion 30b, and the position in the axial direction and the orientation of the retractor hook 50 with respect to the hook support section 40, can be fixed with a small number of operations. Therefore, the labor for retraction can be reduced, and the labor saved by the reduction can be used for the surgical procedure.

In the retractor unit 1 including the retractor 10 fixed on the rib spreader 100, the opening direction of the rib spreader 100 having a shape of inverted letter C with two right-angled corners and the opening direction of the U-shaped support arm 30 are different from each other; namely, the opening between the core frame 110a and the slide frame 120 and the opening between the ends of the extended aim portion 30b are directed in different directions from each other. Therefore, the stopping blades 130 for stopping and widening the surgery area 210, and the retractor hook 50 for holding the incised area 201 in a widened state or holding the sternum 200 in a closed state, do not interfere with each other. Thus, a desired retracted state can be realized.

In addition, the shaft insertion hole 44a is formed to be parallel to the end portions 42 of the U-shaped pedestal. Therefore, in the state where, for example, the hook support section 40 is attached to the support arm 30, unlike when the shaft insertion hole 44a is inclined inward and downward with respect to the support arm 30, even when the hook support section 40 is rotated inward with respect to the support arm 30, a rear end of the base shaft 51 protruding upward is unlikely to be above the fixing and adjusting pinch bolt 45. This prevents the operability of the fixing and adjusting pinch bolt 45 from being lowered.

In the above description, the extended arm portion 30b on both sides of the central fixed portion 30a is formed of a plastically deformable composite member. Alternatively, the extended arm portion 30b may directly extend from the fixing table 20, or both of the central fixed portion 30a and the extended arm portion 30b may be formed of a plastically deformable composite member. In the structure in which the extended arm portion 30b directly extends from the fixing table 20, the fixing table 20 acts as the holder main body.

In the above description, the extended arm portion 30b is entirely formed of a plastically deformable composite member. Alternatively, borders between the central fixed portion 30a and the extended arm portion 30b and a non-end part of the extended arm portion 30b may be formed of a plastically deformable composite member, whereas the other parts may be formed of a metal material substantially the same as that of the central fixed portion 30a.

In addition, the extended arm portion 30b is formed of a plastically deformable composite member having a circular cross-section, which includes the core metal part 31 of lengthy cross-section and the circle-forming resin part 32, and the thickness in the horizontal direction of the core metal part 31 of lengthy cross-section is uniform in a length direction of the extended arm portion 30b. Alternatively, the thickness of the core metal part 31 of lengthy cross-section in the horizontal direction may be gradually decreased as the core metal part 31 of lengthy cross-section approaches the tips of the extended arm portion 30b. Owing to this, when the extended arm portion 30b is plastically deformed in the in-plane direction Fh in the virtual plane F, the extended arm portion 30b has an improved bending performance as approaching the tips thereof, and thus the operability of the extended arm portion 30b can be improved.

The core metal part 31 of lengthy cross-section may be formed of a shape memory alloy, as flexible steel, which is plastically deformed in accordance with the temperature of use. In this case, the shape of the support arm 30 is returned to the memorized shape when being heated by autoclave, and so the convenience is improved.

The elements of the present invention and the elements in the above-described embodiment correspond as follows.

The holder main body of the present invention corresponds to the central fixed portion 30a of the support arm 30;

the arm portion corresponds to the extended arm portion 30b of the support arm 30;
the in-plane plastic deformation means corresponds to the plastically deformable composite member forming the extended arm portion 30b;
the attachment insertion hole corresponds to the arm insertion hole 41a;
the position fixing means corresponds to the fixing and adjusting pinch bolt 45 co-operative with the thread 46a;
the base shaft corresponds to the base shaft 51;
the insertion hole corresponds to the shaft insertion hole 44a;
the plastically deformable portion corresponds to the core metal part 31 of lengthy cross-section; and
the outer portion corresponds to the circle-forming resin part 32. However, the present invention is not limited to the above-described embodiment.

For example, in the above description, the support arm 30 is formed to be U-shaped as seen in a plan view. Alternatively, the support arm 30 may have any shape suitable to the use thereof; for example, the support aim 30 may have a shape of letter C, inverted letter C with two right-angled corners, letter L or the like as seen in a plan view.

The extended arm portion 30b may be provided with a plurality of hinge mechanisms pivotable in a horizontal direction with respect to a vertical pivot axis as in-plane plastic deformation means. In the case where the hinge mechanisms are provided, a ratchet mechanism capable of causing provisional fixation every prescribed rotation angle may be provided.

The central fixed portion 30a and the arm fixing portion 21 may be formed to have a polygonal cross-section. Owing to this, the force of rotating and fixing the support arm 30 with respect to the fixing table 20 can be improved.

In the above description, the extended arm portion 30b is formed of a plastically deformable composite member having a circular cross-section, which includes the core metal part 31 of lengthy cross-section and the circle-forming resin part 32. Instead of the circle-forming resin part 32, a metal member which has a higher bending performance than the core metal part 31 of lengthy cross-section and is durable against repeated deformation may be used. Instead of the core metal part 31 of lengthy cross-section, a resin member which is plastically deformable in repetition may be used.

In the above description, the central fixed portion 30a, in substantially the entirety of the circular cross-section, is formed of the same metal material as that of the core metal part 31 of lengthy cross-section. The central fixed portion 30a is not limited to this, and may be formed of a plastically non-deformable metal material, a resin or the like.

### INDUSTRIAL APPLICABILITY

The present invention is usable as, for example, a surgical tool for thoracotomy.

### REFERENCE SIGNS LIST

10 . . . Retractor
20 . . . Fixing table
30a . . . Central fixing portion
30b . . . Extended arm portion
31 . . . Core metal part of lengthy cross-section
32 . . . Circle-foaming resin part
40 . . . Hook support section
41a . . . Arm insertion hole
44a . . . Shaft insertion hole
45 . . . Fixing and adjusting pinch bolt
46a . . . Thread
50 . . . Retractor hook
51 . . . Base shaft
Fh . . . In-plane direction
Fv . . . Out-of-plane direction

1. A retractor, comprising:
a holder main body;
an arm portion extending on both sides of the holder main body; and
a hook support section, attached to the arm portion so as to be slidable in a longitudinal direction, for supporting a retractor hook;
wherein in at least a part of at least one of the holder main body and the arm portion, in-plane plastic deformation means is provided for permitting plastic deformation in an in-plane direction in a virtual plane formed of the holder main body and the arm portion while restricting deformation in an out-of-plane direction, when receiving a deformation load for causing a bending deformation.

2. A retractor according to claim 1, wherein:
the arm portion is formed to have a circular cross-section; and
the hook support section includes an attachment insertion hole for permitting the arm portion to be attached thereto so as to be rotatable in a circumferential direction around a center of the circular cross-section, and position fixing means for fixing the arm portion at a desired rotation position and a desired slide position.

3. A retractor according to claim 1, wherein:
the hook support section includes an insertion hole for permitting a base shaft included in the retractor hook to be inserted therethrough; and
the position fixing means fixes the base shaft inserted through the insertion hole.

4. A retractor according to claim 2, wherein:
the hook support section includes an insertion hole for pen rifting a base shaft included in the retractor hook to be inserted therethrough; and
the position fixing means fixes the base shaft inserted through the insertion hole.

5. A retractor according to claim 2, wherein:
the in-plane plastic deformation means is formed of a plastically deformable composite member including:
a plastically deformable portion which is located at an inner position in the in-plane direction of the circular cross-section of the arm portion and has a cross-section thicker in the out-of-plane direction than in the in-plane direction; and
an outer portion which is located outer to the plastically deformable portion in the in-plane direction of the circular cross-section and has a higher bending performance than the plastically deformable portion; and
the in-plane plastic deformation means forms at least a part of at least one of the holder main body and the arm portion.

6. A retractor according to claim 3, wherein:
the in-plane plastic deformation means is formed of a plastically deformable composite member including:
a plastically deformable portion which is located at an inner position in the in-plane direction of the circular cross-section of the aim portion and has a cross-section thicker in the out-of-plane direction than in the in-plane direction; and
an outer portion which is located outer to the plastically deformable portion in the in-plane direction of the circular cross-section and has a higher bending performance than the plastically deformable portion; and
the in-plane plastic deformation means forms at least a part of at least one of the holder main body and the arm portion.

7. A retractor according to claim 4, wherein:
the in-plane plastic deformation means is formed of a plastically deformable composite member including:
a plastically deformable portion which is located at an inner position in the in-plane direction of the circular cross-section of the arm portion and has a cross-section thicker in the out-of-plane direction than in the in-plane direction; and
an outer portion which is located outer to the plastically deformable portion in the in-plane direction of the circular cross-section and has a higher bending performance than the plastically deformable portion; and
the in-plane plastic deformation means forms at least a part of at least one of the holder main body and the arm portion.
